(19) United States
(12) Patent Application Publication
(10) Pub. No.: US 2015/0142046 A1
(43) Pub. Date: May 21, 2015

(54) METHOD OF SINUSITIS TREATMENT

(71) Applicants: Roger Andersen, (US); Martin Yvon Desrosiers, (US); Nicolas Loebel, Woodinville, WA (US); Sinuwave Technologies, Inc., Bothwell, WA (US)

(72) Inventors: Roger Andersen, Ladysmith (CA); Martin Yvon Desrosiers, Montreal (CA); Nicolas Loebel, Woodinville, WA (US)

(21) Appl. No.: 14/543,065

(22) Filed: Nov. 17, 2014

Related U.S. Application Data
(60) Provisional application No. 61/905,564, filed on Nov. 18, 2013.

(51) Int. Cl. A61M 25/10

(52) U.S. Cl. CPC A61M 25/10; A61M 2025/105; A61M 2025/107; A61M 2210/0618; A61M 2210/1089; A61M 2202/203; A61M 2202/206; A61M 2202/20; A61M 2202/0014
USPC 606/196; 606/192

(57) ABSTRACT

The present invention provides a method to treat sinusitis, a method for improved medication delivery to the sinus mucosa, and a method for staunching bleeding of the sinus mucosa comprising of comprising compressing sinus mucosa of a sinus of a patient against bone tissue of the sinus to a predetermined level without damaging the bone tissue. The present invention further provides a method to treat nasal polyposis comprising compressing nasal polyps contained within the nasal cavity against its lining to reduce volume of the nasal polyps. The present invention provides a method to treat chronic urethral syndrome comprising compressing interior surfaces of an urethra in order to stimulate and cause excretion out of urethral mucosa located along length of the urethra. The present invention further provides additional methods relating to drug delivery and visualization.

### CLAIM OF BENEFIT OF FILING DATE

This application claims the benefit of the filing date of U.S. Provisional Application Ser. No. 61/905,564 titled: “METHOD OF SINUSITIS TREATMENT” filed on Nov. 18, 2013, which is incorporated herein by reference for all purposes.

### FIELD OF INVENTION

The present invention relates to methods and devices for treating sinusitis, especially chronic recurrent sinusitis using compression of the sinus mucosa in order to express/expel materials from the paranasal sinuses.

### BACKGROUND

Sinusitis is an inflammatory disease of the paranasal sinuses. The paranasal sinuses are connected hollow cavities in the skull. Referring to FIGS. 1-2, these hollow cavities include the following: the maxillary sinus(es) 2, the ethmoid sinus(es) 4, the frontal sinus(es) 6, and the sphenoid sinus(es) 8. The maxillary sinus 2 is located inferior to the eye orbit 10 and lateral to the nasal cavity 12. The ethmoid sinus 4 is located superior to the maxillary sinus 2. The frontal sinus 6 is located superior to the ethmoid sinus 4. The sphenoid sinus 8 is located posterior to the ethmoid sinus and superior to the maxillary sinus 2. The sinuses (2, 4, 6, 8) contain cells that produce mucus which is transported by the mucociliary transport from the sinuses (2, 4, 6, 8) through their openings, known as sinus ostiums, to the nasal cavity 12.

Chronic Rhinosinusitis (“CRS”) is persistent inflammation of the sinuses. The human and economics cost of CRS are substantial. Patients affected with CRS suffer from headache, facial pain, and sinus infections, and experience a reduced quality of life, comparable to that of congestive heart failure and chronic obstructive pulmonary disease. Operations for CRS are among the most common, with over 400,000 surgeries for CRS performed annually in the USA and another 40,000 annually in Canada. The economic burden of CRS on health care resources is considerable. For example in Canada, the direct costs of medication, doctor visits and surgery are assessed at approximately $860 million annually. Productivity costs for CRS patients unresponsive to medical treatment is much more than suspected, and is estimated to add an additional $1.3 billion in lost productivity annually for Canadian society. The substantial cost of lost productivity related to CRS is higher than that associated with diabetes, chronic migraines and severe asthma.

CRS has a prolonged course and is frequently resistant to medical therapy. Current therapeutic strategies involve a combination of nasal irrigations, corticosteroids and antibiotics. Unfortunately, these are rarely curative and raise concerns regarding safety. Antibiotic overuse is a major driver of development of antibiotic resistance. Sinusitis is responsible for 25% of all antibiotic use in humans in North America and is thus believed to contribute significantly to the development of antibiotic resistance. Oral corticosteroid therapy can lead to severe short and long term adverse effects including diabetes, fractures, psychosis, depression, glaucoma and cataracts. In the absence of a response to medical therapy, endoscopic sinus surgery (“ESS”) is indicated. Success is not uniform and up to 30% of patients will continue to manifest signs and symptoms of the disease. Additional therapeutic options for these patients are very limited, as early events leading to the development of CRS remain to be described.

Given the frequency, the human suffering and economic burden of refractory CRS, and the deficiencies in effectiveness and adverse effects of current therapies, novel avenues of therapy for CRS are urgently required. The use of balloon catheters to treat sinus diseases has been disclosed. For example, U.S. Pat. No. 8,114,113 disclosed the use of a balloon catheter to dilate an ostium or duct, or the choana, to create a new opening (ostium) from a sinus to the nasal cavity, and to conduct sinusotomy. However, these methods often cause tissue damage and trauma to the treatment area. Furthermore, restoration of the drainage passage created by these methods does not always lead to resolution of CRS. After all, studies have shown recurrence of CRS in up to 40% of a treated population within twelve months after surgery in which restoration of the drainage passage was achieved.

### BRIEF DESCRIPTION OF THE DRAWINGS

FIG. 1 is a schematic front view of a portion of a human face showing the paranasal sinus and nasal cavity;

FIG. 2 is a schematic lateral cross-sectional view of the paranasal sinuses;

FIG. 3 is a diagrammatic perspective view of a balloon catheter that be used in accordance to a method of the present invention;

FIG. 4 is a close up diagram axially cross-sectional view of a portion the balloon catheter;

FIG. 5 is a close up diagrammatic longitudinally cross-sectional view of a portion the balloon catheter with its deflated balloon;

FIG. 6 is FIG. 1 shown with a portion of the catheter with its inflated balloon placed into the maxillary sinus in accordance to a method of the present invention;

FIG. 7 is a black and white photo of a section of a sinus mucosa at 400× magnification that has been gram stained to illustrate bacteria;

FIG. 8 is a close up diagram axially cross-section view of a portion of h balloon of one embodiment of the balloon catheter;

FIG. 9 is a close up diagram of one embodiment of a portion of the catheter with its inflated balloon placed into the maxillary sinus in accordance to a method of the present invention; and

FIG. 10 is a close up diagram of another embodiment of a portion of the catheter with its inflated balloon placed into the maxillary sinus in accordance to a method of the present invention.

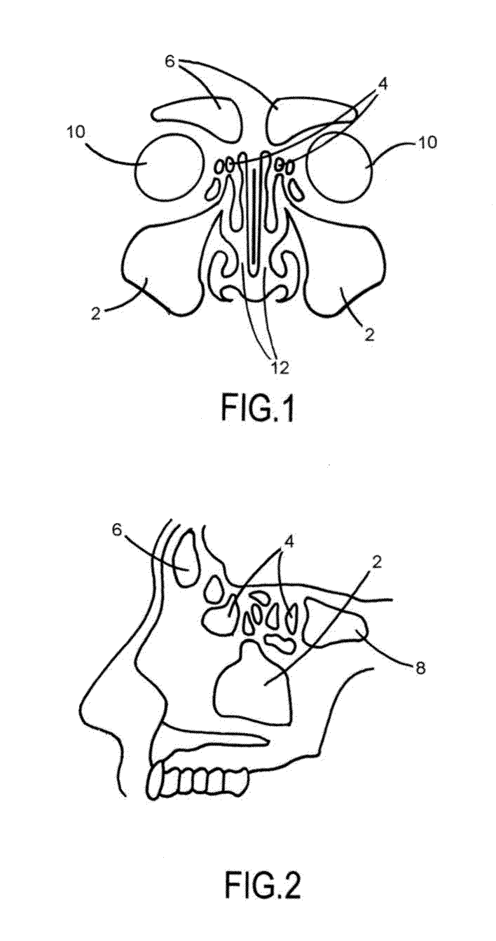

### DETAILED DESCRIPTION OF THE INVENTION

As discussed above, there is a significant subpopulation of patients with CRS who remain resistant to cure despite rigorous treatment regimens including surgery, allergy therapy and prolonged antibiotic therapy. Studies in refractory CRS patients have identified persistent bacterial colonization/infection as important factors in the persistency of CRS. Microbiome studies of the nasal cavity and sinus passages have furnished an underpinning to these observations and extended our understanding by documenting that there is a characteristic microbiota associated with healthy sinuses, which is characterized by a predominance of Gram-positive organisms, including members of the firmicutes and actinobacteria classes. In CRS patients, this microbiome profile is skewed towards an increased quantity of bacteria, with an increase in the relative predominance of Gram-negative organisms, with proteobacteriacae representing the dominant species.

In one study, a population of CRS patients having previously undergone endoscopic sinus surgery for disease management were assessed according to their evolution of disease as either “favorable” (Le., no persistent symptoms or inflammation of the mucosa) vs. “unhealthy” (i.e., symptoms and inflammation of the sinus cavities). Patients with “unhealthy” sinus cavities have severe impairment of quality of life and are characterized by an overall increased bacterial load with a significant overrepresentation of gram-negative species when compared to the “favorable” patients. It is believed that the dysbiotic microbiome contributes towards development and persistence of CRS. In addition, mucus at the sinus mucosal level has been shown to contain disease-causing microorganisms such as bacteria, viruses, and fungi (collectively hereinafter referred to as “pathogen”). The mucus has also been shown to be rich in bacteriophages, small viruses that specifically infect bacteria and cause or aggravate pre-existing CRS. It is thus clear that clearance of disease and restoration of health in the sinuses will require a reduction of the pathogen (especially bacterial) burden at the level of the sinus mucosa. For the purpose of this specification, the term “pathogen-laden” mucus is hereinafter defined as mucus containing at least one microorganism such as bacterium, virus, or fungus.

Reducing pathogen burden within the sinus mucosa is challenging due to the fact that the disease producing bacteria produce a polysaccharide matrix coating (commonly known as a “biofilm”) that (i) protects them from host immune system detection and destruction, and (ii) increases their resistance to antibiotics. Bacteria in this biofilm form are observed to reside within the mucus found on and within the sinus mucosa. Conventional therapies have failed to successfully eradicate the above-discussed biofilm(s) from the sinuses. We believe two factors likely contributed to such failures. First, the inflamed sinus mucosa presents an irregular contour, with numerous folds within and in between area of hyperplastic mucosa serving to “trap” biofilm-containing mucus and isolate it from conventional therapies that treat the “exposed” surface of the sinus mucosa. Second, biofilm-containing mucus is not limited to the surface of the sinus mucosa but exists in other areas deep within the sinus mucosa making it difficult to eradicate by surface therapies.

The sinus mucosa is consisted of an epithelial border containing both epithelial cells and glandular goblet cells. Deep to these structures lie additional glandular structures such as mucous cells and serous cells which excrete mucus onto the sinus mucosal surface. For the purpose of this specification, glandular structures shall hereinafter defined as goblet cells, mucous cells, serous cells, and a combination thereof. This mucus contains substances implicated in airway homeostasis and for defense against pathogens such as lactoferrin, lysozyme, and other anti-microbial proteins. In addition. the mucus coating of the sinus mucosa traps pathogens and foreign material which adhere to the surface of the mucus. In healthy individuals, mucus on the surface of the sinus mucosa is propelled by cilia, small hairs on the epithelial surface, which transport the mucus away towards the sinus openings and then posteriorly to the pharynx for disposal via swallowing. Biofilm containing mucus impairs this sinus mucociliary defense by preventing cilia from being able to clear the mucus from the sinuses.

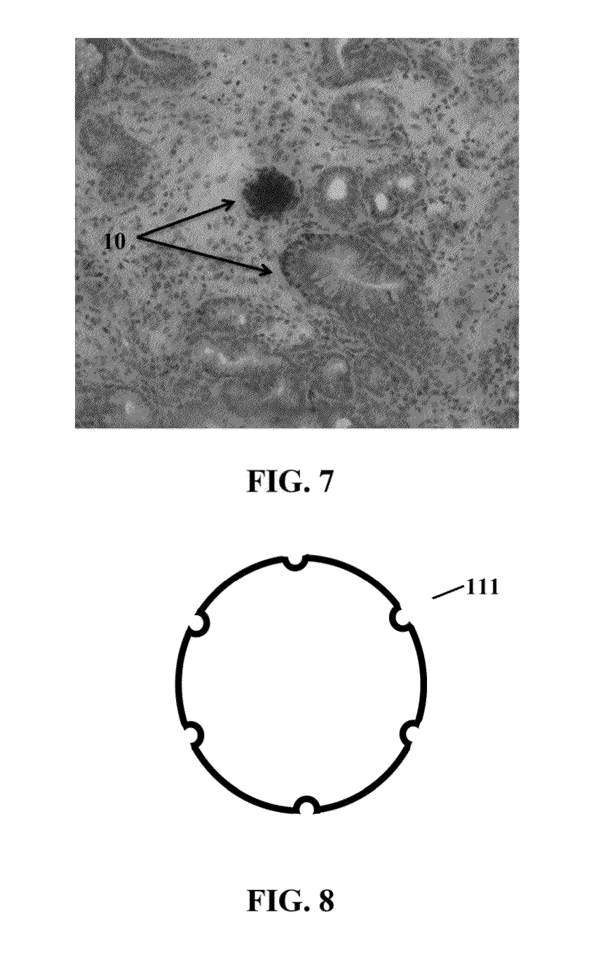

In sinus mucosal biopsies from patients with CRS, glandular structures are hypertrophied and increased in number. Referring to FIG. 7, bacteria (10) are found within the epithelium (shown as dark clumps) and the glandular structure (shown as a line of small dark spots along the bottom of a glandular structure). The biopsies show that glands in the sinus mucosa harbor bacteria. Such bacteria are believed to contribute to CRS.

The present invention provides a method to treat sinusitis, especially CRS, that reduces or even prevents tissue damage and trauma to the treatment area by compressing the sinus mucosa including its glandular structures within a sinus in order to expel pathogen-laden mucus out of the sinus but without damaging (e.g., breaking, fracturing, or the like) the bone tissue of the sinus itself. The method offers clearance of pathogen-laden mucus from the sinus beyond what could be removed by conventional clinical means by displacing the pathogen-laden mucus either trapped in the folds of the sinus mucosa or within the sinus mucosa itself.

In one study, sinus mucosa was removed from a CRS patient and divided into a control section and an experimental section. The sinus mucosa sections were cleaned, pat-dried, and weighed. The experimental section is then placed into a container and subjected to compression by having a balloon applying pressure of 1 atmosphere onto the sinus mucosa squeezing it against the wall of the container (which served as a model of the bony tissue wall of a sinus). After compression, the experimental section was removed, pat-dried, weighted, and examined under a microscope. When compared to the control section, the experimental section showed no physical damage indicating that the compression did not harm the sinus mucosa. The compression caused the experimental section to experience a 16% loss in mass while the control section experienced no loss in mass. The experiment demonstrated that the compression of the sinus mucosa, caused by the pressure exerted from the balloon, was successful in expelling materials from the sinus mucosa including but not limited to its glandular structures.

Compression of the sinus mucosa can be achieved using various means. In one embodiment of the present invention, compression of the sinus mucosa is achieved by inflation of a catheter balloon. The catheter balloon is inserted into the lumen of a sinus cavity (e.g., maxillary, frontal, ethmoid, or sphenoid sinus) using pre-bended catheters, or under direct endoscopic visualization, or using image guided visualization or over a guidewire. Once the balloon is inside the sinus cavity, fluid is then infused into the balloon in order to inflate it within the closed rigid confines of the sinus cavity formed by the bone tissue of the sinus. When the balloon is inflated, it applies a predetermined pressure diffusely to the sinus mucosa against the bone tissue

of the sinus without damaging the bone tissue itself. The balloon basically compresses the sinus mucosa against the rigid bony sinus cavity wall or lining squeezing, expressing, expelling, excreting (collectively hereinafter collectively referred to as “expressing or expression”) materials such as pathogen-laden mucus out of the sinus mucosa. The compression expresses the pathogen-laden mucus from the “exposed” surface of the sinus mucosa, within crypts and folds of the “unexposed” surface of the sinus mucosa, and the interior “reservoirs” of the sinus mucosa glandular structures (including but not limited to the hypertrophied glandular structures). Once the pathogen-laden mucus has been expressed from the sinus mucosa, it can be removed from the sinus through natural sinus ciliary clearance mechanisms which transport the pathogen-laden mucus away towards the sinus openings and then posteriorly to the pharynx for disposal via swallowing. The compression of the sinus mucosa and its resulting expression of the pathogen-laden mucus from the sinus mucosa can generate a healing response by liberating the pathogen-laden mucus from the mucosal tissue, leaving it on top of the “exposed” surfaces of the sinus mucosa whereupon it can be easily removed from the sinus either through natural means, man-made means, or a combination thereof. For example. during compression of the sinus mucosa, the inflated balloon can optionally expresses the pathogen-laden mucus around the edges of the inflated balloon towards the ostium along its natural pathway into the nasal cavity for expression. Upon completion of treatment, the balloon is deflated and any remaining pathogen-laden mucus or debris within the sinus cavity irrigated or aspirated. The compression discussed herein by the balloon catheter reduces the amount of pathogen-laden mucus present within the sinus thereby lowering the pathogen burden within the sinus. This pathogen reduction also leads to a reduction of the amount of toxic products normally produced by the pathogen. The reductions in pathogen and toxic products within the sinus help to resolve inflammation and to restore normalcy to the sinus mucosa. Accordingly, the method of the present invention is an effective way to combat CRS especially compared to the limitations inherent in conventional CRS therapies discussed above. It has been successfully utilized to treat sinusitis including acute and CRS.

In addition to the balloon catheter, the method of the present invention can be achieved using other suitable art-disclosed means to provide the desired compression to the sinus mucosa. For example, in one embodiment, self-inflated material can be placed inside the sinus and allowed to swell to a predetermined size and/or dimension. For the purpose of this specification, self-inflated material shall be defined to include compressed cellulose, sponge, other suitable art-disclosed self-inflated device (e.g., see U.S. Patent Application Publication No. 2014/0031852), and a combination thereof. The swelling process can be achieved either through natural means such as osmotic action within the sinus or man-made means such as fluid irrigation into the sinus. As noted above, the provided compression must be at a level that allows the expulsion of pathogen-laden mucus from the sinus mucosa without damaging the (bone) tissue of the sinus itself. For example, the pressure applied to the sinus mucosa can range anywhere from about 0.1 atm to about 2.5 atm, from about 0.2 atm to about 2 atm, from about 0.25 atm to about 1 atm.

In one embodiment, medication can optionally be placed before, during or after the compression treatment. For the purpose of this specification, medication is hereinafter defined as one or more compounds desired to be placed into the sinus for medical treatment of patient including but not limited to anti-inflammatory drugs, antibiotics, regenerative drugs or tissues, wound modulating agents, bacteria with probiotic properties (i.e., healthy bacteria), hemostatic agents, and a combination thereof. The medication can be in any suitable art-disclosed form such as a fluid (including gel) or on the surface of or incorporated within a resorbable/non-resorbable membrane placed inside the sinus.

Moreover, if the medication is to be applied during compression of the sinus mucosa, it can be applied in the form of a coating (e.g., gel, film, polymer matrix, or the like) on the surface of the balloon. Upon inflation of the balloon, the medication will be deposited and/or adhere to the sinus mucosa. During compression of the sinus mucosa, the increased pressure on the sinus mucosa will increase deposition and/or penetration of the medication into the sinus mucosal tissue beyond that realizable by topical application alone. Accordingly, this additional benefit of better medication delivery and penetration is achieved by the present invention if the medication is applied either before or during the compression of the sinus mucosa. In one embodiment, the present invention of compressing the sinus mucosa is used for the purpose of delivering medication to the sinus mucosa in a more effective manner. This medication delivery method of the present invention can be performed alone by itself or in conjunction with other purposes of the present invention (e.g., removing pathogen-laden mucus from the sinus, staunching bleeding during surgery, etc.).

In another embodiment, the medication is contained within the inflated balloon and the inflated balloon is constructed of biodegradable material having micropores which allow the medication to be dispensed into the sinus mucosa over a predetermined amount of treatment time. Thereafter, the inflated balloon is left inside the sinus until it degrades or is removed by the physician. Depending upon the desired application, the inflated balloon can remain in the sinus for a predetermined amount of time ranging from month(s), week(s), day(s), hour(s), and minute(s). For example, in one embodiment the inflated balloon is placed into the sinus for a period ranging from about 1 day to about 4 weeks.

In light of the foregoing, the present invention also includes an improved method of medication delivery via compression of a targeted treatment area other than the sinus mucosa. The compression of the targeted treatment area can be achieved by any suitable art-disclosed means including but not limited to the inflated balloon 110 and/or other inflatable devices and procedures discussed above relating to compression of the sinus mucosa. The targeted treatment area can be any suitable treatment area wherein it is desired for better deposition and/or penetration of medication within such treatment area. Examples of the targeted treatment area includes but is not limited to any mucosal tissue within the body (e.g., mucosa surface/lining of the urethra or the like), nasal polyps contained within the nasal cavity, or the like.

The method of the present invention offers a minimally invasive means of removing pathogen-laden mucus and/or other materials from sinus so can thus be conducted outside of the operating room in a clinic or outpatient setting. Furthermore, it can be performed as a stand-alone procedure or in conjunction with other procedures such as endoscopic sinus surgery, balloon sinuplasty or the like. When the method is performed in conjunction with surgical or other procedures that may involve bleeding of the sinus mucosa, compression of the sinus mucosa will provide the benefit of producing a hemostatic effect on the sinus mucosa by staunching any potential bleeding. In fact, the present invention of compressing of the sinus mucosa can be used for this sole purpose or in conjunction with other purposes of the present invention (e.g., removing pathogen-laden mucus from the sinus, drug delivery, etc.). In one embodiment, the compression of the sinus mucosa is used during or after sinus surgery in order to deliver medication to the sinus mucosa and to staunch any bleeding of the sinus mucosa, In this embodiment, the medication delivered includes but is not limited to a hemostatic agent.

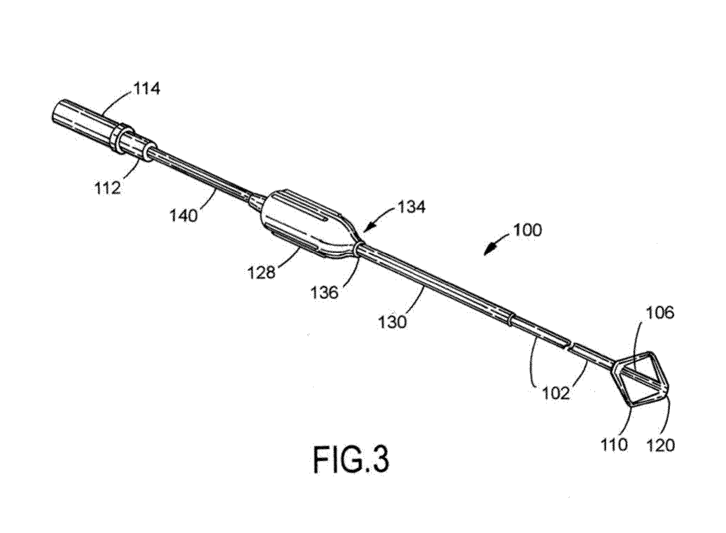

Referring to FIGS. 3-5, a balloon catheter 100 to be used in the present invention has an outer tube 102 with an outer lumen 104, an inner tube 106 with an inner lumen 108, an inflatable balloon 110, and a fluid connector 112 . At least a portion of the inner tube 106 is located within the outer lumen 104 and the inner tube 106 and the outer tube 102 are optionally coaxial. The outer tube 102 and the inner tube 106 are constructed of flexible materials so that they can flex, bend, and conform to the passageways of the nasal cavity, ostium(s), and sinus(es). The outer tube 102 also provides structural support to the catheter 100. The outer tube 102 can be constructed of any art-disclosed material suitable to provide support and flexibility such as aluminum alloy or the like. The inner tube 106 is constructed of any art-disclosed materials suitable to provide flexibility and conformability such as plastic tubing, polyurethane tubing, silicone tubing, Tygon® tubing, or the like.

The connector 112 is located at proximate end of the catheter 100 and connects the catheter 100 to a fluid source 114. The connector 112 can be any suitable art-disclosed connector or connectors (e.g., luer lock(s), ferrule(s), or the like). The connector 112 is connected to proximate end of the inner tube 106 and during operation is in fluid communication with the inner lumen 108. When the connector 112 is connected to the fluid source 114, the inner lumen 108 is in fluid communication with the fluid source 114 and can deliver (i) fluid from the fluid source 114 to the balloon 110 for inflation of the balloon 110; (ii) and/or from the balloon 110 to the fluid source 114 for deflation of the balloon 110. The present invention also contemplates that to deflate the balloon 100, fluid contained within the balloon 110 can be alternatively transported from the balloon 110 out of the catheter 100, via the inner lumen 108 and the connector 112, into other disposal means (i.e., not the fluid source 114). Fluid from the fluid source 114 can be any suitable fluids such as liquids (e.g., water, saline solution, gels etc.) and gas (e.g., air).

Referring to FIG. 5, the balloon 110 is attached to the distal region 116 of the inner tube 106. The distal region 116 may contain port(s) 118 that transport fluid in and out of the balloon 110 during its inflation and deflation processes. An optional capped means 120 is connected to the distal end 122 of the balloon 110 and the distal end 124 of the inner tube 106. In an alternative embodiment, the inner tube 106 terminates near the proximate end 126 of the balloon 110 so no additional port or capped means is needed. In this fashion, the interior of the balloon 110 remains in fluid communication with the inner tube 106 during operation but the inner tube 106 extends very little, if at all, into the interior of the balloon 110.

During operation, the balloon 110 is in fluid communication with the inner lumen 108. The balloon 110 is constructed of resilient biocompatible material such as urethane, polyethylene terephthalate, or the like. Depending on the size and shape of the sinus(es) designated for treatment (“sinus treatment area”), the balloon 110 can be any size and shape suitable for the sinus treatment area. For example, the balloon 110 can be generally ellipsoidal in shape, spherical with tapered ends, cylindrical with tapered ends, or the like. The diameter, length, and total volume of the inflated balloon 110 depends upon the anatomy of the patent's paranasal sinuses. For example, the diameter of the inflated balloon 110 may range from about 2 mm to about 25 mm, from about 2 mm to about 15 mm, and from about 3 mm to about 10 mm or the like. The length of the inflated balloon may range from about 5 mm to about 50 mm, from about 9 mm to about 40 mm, and from about 20 to about 30 mm. The deflated balloon 110 has a size and shape that can gain access to the treatment site.

Referring to FIG. 8 and in one embodiment of the present invention, the balloon 110 may optionally include exterior channels 111 located longitudinally along the surface of the balloon 110. The channels 111 serve as an additional mean to guide the pathogen-laden mucus away from the mucosal surface towards the area for expression. The channels 111 can be any suitable numbers and dimensions. For example, in one embodiment, the balloon 110 includes six channels 111 and each of the channels 111 has approximately the same length as the longitudinal length of the balloon, a width ranging from about 1 to 2 mm, and a depth from about 1 to 1.5 mm. The term “about” as used in this specification is defined as ±5%.

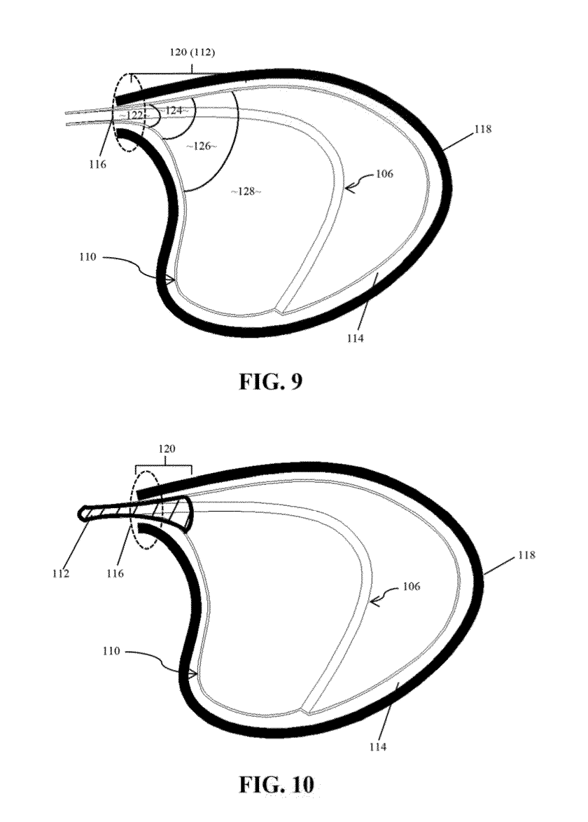

Referring to FIG. 9 and in another embodiment of the present invention, the balloon 110 includes shape restriction feature 112 in order to ensure the balloon 110 is inflated (e.g., with the inner tube 106) in a fashion that it only provides compression of specific desired area(s) within the sinus cavity 114. For example, the shape restriction feature 112 will ensure certain area(s) in or near the sinus ostium (e.g., ostium 116 or the like) is not compressed by the inflated balloon 110. The shape restriction feature 112 can also prevent the inflated balloon 110 from expanding or bulging backwards in a retrograde fashion out of the ostium 116 of the targeted sinus 118. Restricting the inflation of the balloon 110 in the shape restricted portion may also increase its ability to apply peripheral pressure elsewhere in the sinus cavity 114 for any given instillation volume. For example, when the shape restriction feature 112 restricts the inflated balloon 110 from compressing the ostium 116 and/or its surrounding area(s), the inflated balloon 110 is prevented from bulging backwards out of the ostium 116 in a fashion not useful to apply peripheral pressure in the desired portion(s) within the sinus cavity 114 (e.g., the sinus mucosa located within the sinus cavity that is distal from the ostium 116 or the like).

The shape restriction feature 112 can be any suitable art-disclosed means that will restrict the dimensions of the inflated balloon 110 to a desired shape. For example and referring to FIG. 9, the shape restriction feature 112 can be the variable thickness or the variable material density of the balloon 110 wherein shape restricted portion(s) (e.g., the proximate portion 120 or the like) of the balloon 110 includes a gradient of thicknesses or material densities (e.g., from a portion of very high stiffness 122 to a portion of high stiffness 124 to a portion of low stiffness 126 to a portion of lower stiffness 128) that restricts and controls the inflation of the targeted portion(s) of the balloon 110 causing the balloon 110 to inflate into a desired shape which does not compress any area(s) desired for non-compression. Similarly, the shape restriction feature 112 can be variable materials of the balloon 110; coating(s), layer(s), or infused or solvent-swelled additives added to the balloon 110 material in order to restrict and control the inflation of the shape restricted portion(s) of the balloon 110 causing the balloon 110 to inflate into a desired shape. The shape restriction feature 112 can also be a treatment(s) to the balloon 110 material such as radiation-induced crosslinking, thermal treatment, or the like designed to restrict and control the inflation of the shape restricted portion(s) of the balloon 110 causing the balloon 110 to inflate into a desired shape. Finally and referring to FIG. 10, the shape restriction feature 112 can alternatively be a fixed sleeve or a slide-ably movable sleeve that can be placed over a specific portion of the balloon 110 in order to restrict and control the inflation of the shape-restricted portion(s) of the balloon 110 causing the balloon 110 to inflate into a desired shape. The sleeve can be constructed of any suitable art-disclosed material such as biocompatible polymeric material or the like.

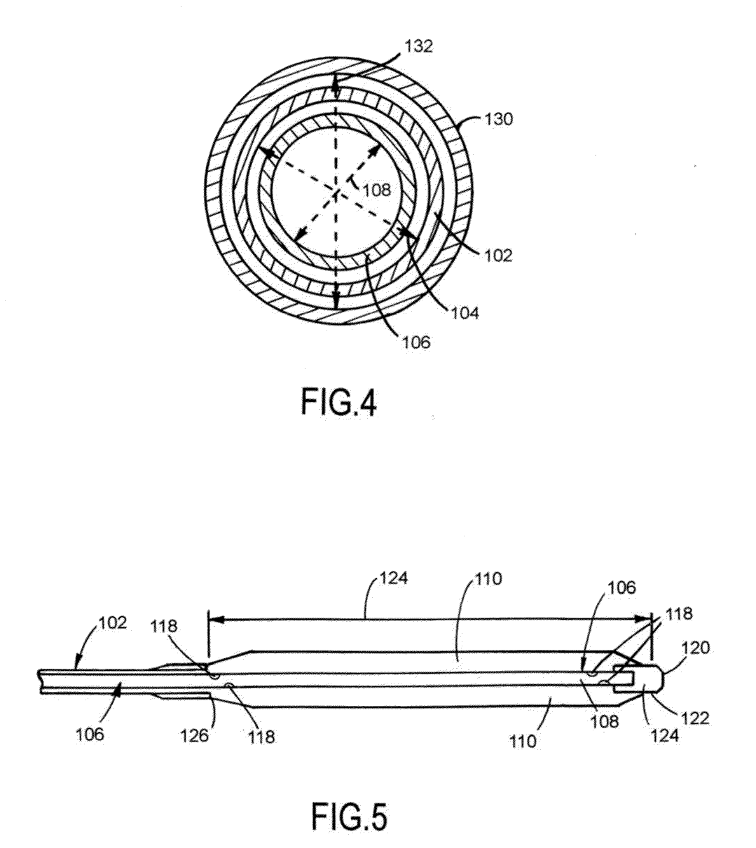

Referring to FIGS. 3-5, the catheter 100 may optionally include a handle hub 128, a support tube 130 having a support lumen 132. Referring to FIG. 4, which shows a close up cross-sectional view of the catheter 100 at a location near the distal end 134 (see FIG. 3) of the handle hub 118, a portion of the outer tube 102 containing a portion of the inner tube 106 are contained within the support lumen 122. It is optional that the outer tube 102, the inner tube 106 and the support tube 130 are all coaxial. The support tube 120 provides stiffness to the catheter 100. The support tube 120 can be constructed of any art-disclosed material suitable to provide the desired stiffness to the catheter 100 such as stainless steel, other metal alloys, or the like.

Referring to FIG. 3, it is optional that the proximate end of the outer tube 130 and the proximate end 138 of the support tube 130 are connected to the handle hub 128 and a protective tube 140 with a protective lumen (not shown) serves as the conduit for fluid communication between the connector 112 and the handle hub 128. A portion of the inner tube 106 is contained within the protective lumen. In an alternative embodiment, the proximate end 142 of the inner tube 106 is connected to the handle hub 128.

Other art-disclosed balloon catheters may be used in accordance to the present invention, including but not limited to, the balloon catheters disclosed in U.S. Pat. No. 8,114,113. In an effort to avoid unnecessary trauma to the patient, it may be desired that the diameter of the catheter 100 that enters the nasal cavity and into the any of the sinuses be sized sufficiently small in order to fit into the nasal passages including the ostiums. In one embodiment, the ostiums are not opened nor enlarged by the catheter 100. Instead, the ostiums are either (i) have been previously opened or enlarged via standard surgical procedures or (ii) naturally sized for entry of the catheter 100.

To treat sinusitis, including acute sinusitis and CRS, the clinician normally identifies a particular sinus of interest to treat via CT, endoscopic evaluation and patient report. Once the sinus of interest has been identified, the clinician may optionally open or enlarge the ostium to such sinus via a standard uncinectomy, antrostomy, ethmoidectomy, sphenoidotomy or frontal sinusotomy procedure, and the patient allowed to heal. Alternatively, this option step of opening the ostium may be omitted and the clinician proceeds directly to the method of the present invention.

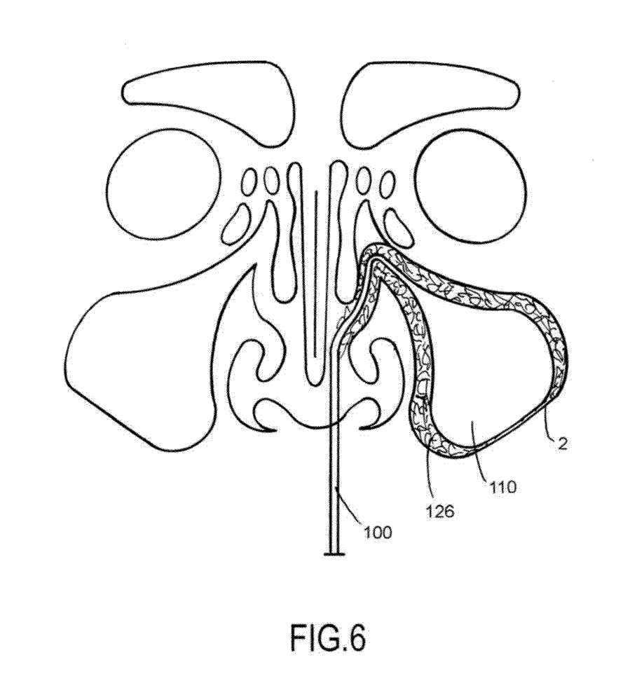

In one embodiment, the method of the present invention includes the first step of inserting a portion of the catheter 100 having the balloon 110 in a deflated state endoscopically into a sinus (e.g., the maxillary sinus 2, the ethmoid sinus 4, the frontal sinus 6, or the sphenoid sinus) through the sinus' ostium (such as the maxillary ostium 136 shown on FIG. 6). The inserted portion of the catheter 100 is sufficiently flexible to allow the clinician to appropriately bend it to allow for easy insertion into the sinus. Bending of the inserted portion of the catheter 100 is performed prior to endoscopic insertion into the sinus. It is optional that a soft, thin, flexible guidewire is first endoscopically placed into the sinus (e.g., 136) using conventional methods. The inserted portion of the catheter 100 is then placed into the desired location within the sinus by being attached to (e.g., threaded over or the like) and advanced over the guidewire. It is optionally to rinse the sinus' ostium using conventional means prior to insertion of the catheter 100 to remove as much dislodgeable contents as possible from the ostium.

Once the balloon 110 is placed into the sinus endoscopically, the second step of the present invention includes inflating the balloon 110 with fluid from the fluid source 114 via the port(s) (or alternatively the distal end 124) of the inner tube 124 under endoscopic visualization until the balloon 110 inflates and expand pressure on mucosal surfaces of the sinus (e.g., filling up the space or cavity within the sinus). The balloon 110 may optionally inflate to a point that it just begins to bulge out of the sinus ostium. The catheter 100 at this stage is connected to the fluid source 114 via the connector 112 so that fluid from the fluid source 114 is in fluid communication with the balloon 112. Referring to FIG. 6, the inflated balloon 110 compresses any lumenal contents of the sinus such as the sinus mucosa and by virtue of its designed shape, the inflated balloon 110 may also optionally expresses at least a portion of such lumenal contents under direct pressure from inside the sinus cavity towards ostium and out of the ostium which shall be defined as lumenal expression. The lumenal expression expresses out some portion of lumenal contents. The lumenal contents include but are not limited to pathogen-laden mucus, bacterial biofilms and/or other waste materials. Please note that neither the sinus' bone tissue that forms the sinus cavity nor the sinus' associated ostium itself is dilated to any appreciable degree. In one embodiment, the sinus hone tissue and ostium experience no dilation. For example, protection of the sinus' bone tissue and ostium from dilation or compression can be achieved by the shape restriction feature 112 of the balloon 110 discussed above. The inflated and expanding balloon 110 places pressure on the sinus mucosal surfaces which is of value in compressing small crypts and mucosal folds otherwise impossible to express or clean via irrigation with fluid. The inflated and expanded balloon 110 may be placed inside the sinus for a period of time from which the clinician considers is necessary to sufficiently express the pathogen-laden mucus from the sinus mucosa (and optionally a desired amount of lumenal expression). For example, this period may range for about 1 minute to 15 minutes, from about 1 minute to 10 minutes, from about 1 minute to five minutes, and from about 2 minutes to 3 minutes.

Once the clinician feels that the treated sinus mucosa has been sufficiently compressed including any optional lumenal expression, the third step of the present invention includes deflating the balloon 110 by transporting the fluid contained within the balloon 110 back out of the catheter 100 to either the fluid source 114 or other disposal means. The fourth step of the present invention includes removing the inserted catheter 100 from the patient. In one embodiment, the inflated balloon 110 itself is detached from the catheter 100 and left inside the sinus for an extended period of time before its removal either through a separate procedure or natural degradation. Depending upon the desired application, the inflated balloon can remain in the sinus for a predetermined amount of time ranging from month(s), week(s), day(s), hour(s), and minute(s). For example, in one embodiment the inflated balloon is placed into the sinus for a period ranging from about 1 day to about 4 weeks.

It should be noted that after the removal of the catheter 100 from the patient, the expressed lumenal contents continue to come out of the treated ostium towards the nasal septum into the osteomeatal complex and out of the sinus cavity. It is optionally to rinse the treated ostium using conventional means after the removal of the catheter 100 to further assist the removal of lumenal contents. The method of the present invention set forth above can be repeated to treat the remaining affected paranasal sinuses.

Another method of the present invention is for the treatment of nasal polyposis. The method includes the same steps as described above for the compression of the sinus mucosa except that instead of compression of the sinus mucosa, compression of the nasal polyps contained within the nasal cavity is performed to reduce the volume of the nasal polyps within the nasal cavity. Pressure is exerted to compress the nasal polyps against the lining of the nasal cavity in order to reduce the volume of the nasal polyps. Reducing the volume of nasal polyps in the nasal cavity provides relief of nasal obstruction in patients with nasal polyposis. This method can be performed as part of the preparation of the nasal cavity for surgery in patients with nasal polyposis, or as a primary therapy for treatment of obstructive symptoms. Reduction of nasal polyps with the nasal cavity prior to nasal surgery improves visualization of the nasal cavity during surgery making the surgical procedure safer and easier. Reduction of volume of nasal polyps improves airflow and relieves symptoms of nasal obstruction associated with CRS with nasal polyps.

Another method of the present invention is for the treatment of chronic urethral syndrome. Chronic urethral syndrome causes patients to have a constant feeling of urgency to void and irritation of the urethra. This method of the present invention includes the first step of inserting a portion of the catheter 100 having the balloon 110 in a deflated state endoscopically into a patients urethra. The inserted portion of the catheter 100 is sufficiently flexible to allow the clinician to appropriately bend it to allow for easy insertion into the urethra. Bending of the inserted portion of the catheter 100, if desired, is performed prior to endoscopic insertion into the urethra. It is optional that a soft, thin, flexible guidewire is first endoscopically placed into the urethra (e.g., 136) using conventional methods. The inserted portion of the catheter 100 is then placed into the desired location within the urethra by being attached to (e.g., threaded over or the like) and advanced over the guidewire.

Once the balloon 110 is placed into the urethra (either directly, endoscopically, or the like), the second step of the present invention includes inflating the balloon 110 with fluid from the fluid source 114 via the port(s) (or alternatively the distal end 124) of the inner tube 124 (optionally under endoscopic visualization) until the balloon 110 inflates and expand pressure on mucosal surfaces and tissues of the urethra (collectively hereinafter referred to as “urethral mucosa:”) by filling up the space or cavity within the urethra. The catheter 100 at this stage is connected to the fluid source 114 via the connector 112 so that fluid from the fluid source 114 is in fluid communication with the balloon 112. The inflated balloon 110 compresses the urethral mucosa, including but not limited to the interior or lumenal surfaces of the urethra and the periurethral glands. Similar to the sinus mucosa, the urethral mucosa also contains disease-causing microorganisms such as bacteria, viruses, and fungi (“pathogen”) and the pathogen can be in a biofilm form. The compression of the urethral mucosa by the inflated balloon 110 expresses such pathogen from the urethral mucosa. The compression also causes stimulation and expression of pathogen and other fluids (hereinafter collectively referred to as “urethral excretion”) from the urethral mucosa located along the length of the urethra. The urethral mucosa includes periurethral glands and compression of the periurethral glands contributes to the expression of urethral excretion. Removal of the urethral excretion from the urethral mucosa relieves symptoms of chronic urethral syndrome. The compression also disrupts the urethral mucosa causing an increase in the amount of white blood cells within the compressed area of the urethral mucosa thus promotes further healing.

In another embodiment, the method described above further includes applying pressure to the urethral mucosa including but not limited to the periurethral glands via the anterior vaginal wall. The application of pressure can be accomplished by using digital pressure along the length of the urethra through the vaginal wall. In this situation, the inflated balloon 110 provides a soft backboard against which the tissues between the urethra and vaginal walls are compressed. This further facilitates the expression of any glandular contents and promotes and inflammatory (i.e., healing) response.

The inflated and expanded balloon 110 may be placed inside the urethra for a period of time from which the clinician considers is desirable to relief symptoms of chronic urethral syndrome. For example, this period may range for about 1 minute to 15 minutes, from about 1 minute to 10 minutes, from about 1 minute to five minutes, and from about 2 minutes to 3 minutes. Once the clinician feels that the treated urethra has been sufficiently compressed, the third step of the present invention includes deflating the balloon 110 by transporting the fluid contained within the balloon 110 back out of the catheter 100 to either the fluid source 114 or other disposal means. The fourth step of the present invention includes removing the inserted catheter 100 from the patient.

The present invention also includes a method of improved visualization of a targeted treatment area using an inflated transparent balloon such as the balloon 110 discussed above. The method includes inserting a balloon near the targeted treatment area and inflating the balloon 110 with fluid. These steps are same as the steps described above for the method for treatment of sinusitis. Once the balloon 110 has been inflated, an endoscope is placed in physical contact with the surface of the inflated balloon 110 in order to see through the inflated balloon 110. Seeing through the inflated balloon 110 provides additional angles of visualization furnished by refraction from the fluid located within the inflated balloon 110. It provides an enhanced line of view from refraction. The endoscope can be a separate device from the balloon catheter 100 or is constructed and incorporated as a part of the balloon catheter 100 using art-disclosed means.

The particulars shown herein are by way of example and for purposes of illustrative discussion of the invention only and are presented in the cause of providing what is believed to be the most useful and readily understood description of the principles and conceptual aspects of the invention. In this regard, no attempt is made to show structural details of the invention in more detail than is necessary for the fundamental understanding of the invention, the description taken with the drawings making apparent to those skilled in the art how the several forms of the invention may be embodied in practice. It is understood that the present invention as described and claimed herein can be used for many additional purposes, therefore the invention is within the scope of other fields and uses and not so limited. The explanations and illustrations presented herein are intended to acquaint others skilled in the art with the invention, its principles, and its practical application. Those skilled in the art may adapt and apply the invention in its numerous forms, as may be best suited to the requirements of a particular use. Accordingly, the specific embodiments of the present invention as set forth are not intended as being exhaustive or limiting of the invention. The scope of the invention should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes.

What is claimed is:

1. A method to treat sinusitis comprising compressing sinus mucosa of a sinus of a patient against bone tissue of the sinus to a level that causes expression of pathogen-laden mucus from the sinus mucosa without damaging the bone tissue wherein the method reduces the amount of pathogen within the sinus.

2. The method of claim 1 wherein the pathogen-laden mucus is expressed from (i) exposed surface of the sinus mucosa: (ii) crypts and folds of unexposed surface of the sinus mucosal: and glandular structures of the sinus mucosa.

3. The method of claim 1 wherein the pathogen-laden mucus is expressed out of the sinus through ostium of the sinus through natural sinus ciliary clearance mechanisms.

4. The method of claim 1 wherein the sinus is selected from the group consisted of a maxillary sinus, frontal sinus, ethmoid sinus, and sphenoid sinus.

5. The method of claim 1 wherein the glandular structures of the sinus mucosa are selected from the group consisting of goblet cells, mucous cells, serous cells, and a combination thereof.

6. The method of claim 1 wherein the pathogen-laden mucus contains pathogen selected from the group consisting of bacteria, viruses, fungi, and a combination thereof.

7. The method of claim 1 wherein the pathogen-laden mucus contains bacterial biofilm.

8. The method of claim 1 wherein the pathogen-laden mucus contains bacteriophage.

9. The method of claim 1 wherein the sinus mucosa is compressed for about 1 minute to about 15 minutes.

10. The method of claim 1 wherein the compression of the sinus mucosa accomplished by the following;
(a) inserting a portion of a catheter having a balloon in a deflated state endoscopically into the sinus through the ostium;
(b) inflating the balloon of the catheter with fluid from a fluid source under endoscopic visualization until the balloon inflates and exerts pressure upon the sinus mucosa causing pathogen-laden mucus to be expressed out of the sinus mucosa
(c) deflating the balloon by transporting the fluid contained within the balloon back out of the catheter; and
(d) removing the catheter from the patient.

11. The method of claim 10 wherein the balloon when inflated also expresses a portion of the pathogen-laden mucus out of the sinus through the ostium and the balloon further includes longitudinal channels along exterior surface of the balloon to assist in guiding the pathogen-laden mucus away from the exposed surface of the sinus mucosa and towards the ostium for expression out of the sinus.

12. The method of claim 10 wherein the balloon includes a shape restriction feature that prevents compression of the ostium by the balloon when inflated.

13. The method of claim 10 wherein (i) a coating of medication is placed upon exterior surface of the balloon; (ii) upon inflation of the balloon, the medication is delivered to the sinus mucosa.

14. The method of claim 1 wherein the compression of the sinus mucosa is accomplished by placing a compressed self-inflated material into the sinus that expands within the sinus and compresses the sinus mucosa.

15. The method of claim 1 wherein the compression of the sinus mucosa is accomplished by the following:
(a) inserting a portion of a catheter having a balloon in a deflated state endoscopically into the sinus through the ostium;
(b) inflating the balloon of the catheter with a fluid from a fluid source under endoscopic visualization until the balloon inflates and exerts pressure upon the sinus mucosa causing pathogen-laden mucus to be expressed out of the sinus mucosa ;
(c) detaching the inflated balloon from the catheter allowing the inflated balloon to remain inside the sinus; and
(d) removing the catheter from the patient wherein the inflated balloon remains inside the sinus for the predetermined time until removal.

16. The method of claim 15 wherein (i) the fluid contains medication; (ii) the inflated balloon is constructed of biodegradable material containing micropores for dispensing the medication into the compressed sinus mucosa over a predetermined amount of time.

17. A method of improved medication delivery to mucosal tissue comprising:
(a) applying medication to the mucosal tissue; and
(b) compressing the mucosal tissue against its adjacent structure to a level that causes disposition of the medication onto the mucosal tissue and penetration of the medication into the mucosal tissue, without damaging the structure.

18. The method of claim 17 wherein the compression of the mucosal tissue is accomplished by the following:
(a) inserting a portion of a catheter having a balloon in a deflated state endoscopically adjacent to the mucosal tissue;
(b) inflating the balloon of the catheter with a fluid from a fluid source under endoscopic visualization until the balloon inflates and exerts pressure upon the mucosal tissue wherein the mucosal tissue is compressed to a level that causes disposition of the medication onto the mucosal tissue and penetration of the medication into the mucosal tissue, without damaging the structure;
(c) deflating the balloon by transporting the fluid contained within the balloon back out of the catheter; and
(d) removing the catheter from the patient.

19. The method of claim 17 wherein the method is accomplished by the e following:
(a) inserting a portion of a catheter having a balloon in a deflated state endoscopically next to the mucosal tissue; and
(b) inflating the balloon of the catheter with a fluid containing the medication from a fluid source under endoscopic visualization until the balloon inflates and exerts pressure upon the mucosal tissue wherein (i) the inflated balloon includes micropores for dispensing the medication into the compressed mucosal tissue over a predetermined amount of time; and
(ii) the inflated balloon remains inside the sinus for the predetermined time until removal.

20. A method to staunch bleeding of the sinus mucosa comprising:
(a) inserting a portion of a catheter having a balloon in a deflated state endoscopically into the sinus through the ostium;
(b) inflating the balloon of the catheter with fluid from a fluid source under endoscopic visualization until the balloon inflates and exerts pressure upon the sinus mucosa;
(c) deflating the balloon by transporting the fluid contained within the balloon back out of the catheter; and
(d) removing the catheter from the patient.

21. The method of claim 20 wherein a coating of medication comprising of a hemostatic agent is placed upon exterior surface of the balloon and upon inflation of the balloon, the medication is delivered to the sinus mucosa.

22. A method to treat nasal polyposis comprising compressing nasal polyps contained within the nasal cavity against lining of the nasal cavity to a level that causes reducing of volume of the nasal polyps.

23. The method of claim 22 wherein the compression of the nasal polyps is accomplished by the following:
(a) inserting a portion of a catheter having a balloon in a deflated state endoscopically into the nasal cavity;
(b) inflating the balloon of the catheter with fluid from a fluid source under endoscopic visualization until the balloon inflates and expand pressure upon the nasal polyps causing materials to be expressed out of the nasal polyps and out of the nasal cavity;
(c) deflating the balloon by transporting the fluid contained within the balloon back out of the catheter; and
(d) removing the catheter from the patient.

24. A method to treat chronic urethral syndrome comprising compressing interior surfaces of an urethra of a patient in order to stimulate and express excretion out of urethral mucosa located along length of the urethra wherein the excretion includes excretion coming out of periurethral glands of the urethral mucosa.

25. The method of claim 24 wherein the excretion includes pathogen selected from the group consisting of bacteria, viruses, fungi, and a combination thereof.

26. The method of claim 24 wherein the excretion includes bacterial biofilm.

27. The method of claim 24 wherein the compression of the interior surfaces of the urethra is accomplished by the following:
(a) inserting a portion of a catheter having a balloon in a deflated state endoscopically into the urethra;
(b) inflating the balloon of the catheter with fluid from a fluid source under endoscopic visualization until the balloon inflates and expand pressure on the interior surfaces of the urethra, compresses the interior surfaces of the urethra in order to stimulate and cause excretion of the urethral mucosa along length of the urethra;
(c) deflating the balloon by transporting the fluid contained within the balloon back out of the catheter; and
(d) removing the catheter from the patient.

28. A method for improved visualization of a treatment area comprising:
(a) inserting a portion of a catheter having a balloon in a deflated state endoscopically adjacent to the treatment area;
(b) inflating the balloon of the catheter with a fluid from a fluid source under endoscopic visualization;
(c) visualizing the target area endoscopically by placing an endoscope in direct physical contact with the inflated balloon.
